(19) United States
(12) Patent Application Publication
(10) Pub. No.: US 2007/0167658 A1
(43) Pub. Date: Jul. 19, 2007

(54) Process for producing alkyl aromatic hydroperoxide

(75) Inventors: Mitsuru Onuma, Ichihara-shi (JP); Tomoyuki Noritake, Ichihara-shi (JP); Masaru Ishino, Sodegaura-shi (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED, Tokyo (JP)

(21) Appl. No.: 11/652,592

(22) Filed: Jan. 12, 2007

(30) Foreign Application Priority Data
Jan. 18, 2006 (JP) 2006-009616
Nov. 28, 2006 (JP) 2006-319697

(51) Int. Cl. C07C 409/00

(52) U.S. Cl. 568/558

(57) ABSTRACT

A process for producing an alkyl aromatic hydroperoxide, which comprises oxidizing an alkyl aromatic compound with an oxygen-containing gas in the presence of a water-soluble iron compound, wherein a concentration of the iron compound in the oxidation reaction system is 0.0001 to 10 ppm by weight in terms of iron metal.

### BACKGROUND OF THE INVENTION

1. Field of the Invention

The present invention relates to a process for producing an alkyl aromatic hydroperoxide. For example, cumene hydroperoxide obtained by oxidation of cumene is used for industrial production of phenol, and ethylbenzene hydroperoxide obtained by oxidation of ethylbenzene is used for production of propylene oxide by a Halcon process. Further, diisopropyl benzene hydroperoxides obtained by oxidation of diisopropyl benzene are useful as a raw material for resorcinol or hydroquinone production.

2. Description of Related Arts

In a process for producing an alkyl aromatic hydroperoxide by oxidizing an alkyl aromatic hydrocarbon with air, an addition of various catalysts for accelerating the oxidation reaction rate has been studied. For example, the addition of a metal compound such as a transition metal complex salt catalyst (e.g. JP8-245568 A), a polyvalent amine metal complex catalyst (e.g. JP2000-119247 A) or a transition metal compound catalyst supported on active carbon (e.g. JP8-259529 A) is known. Further, an example in which a transition metal compound is used as a co-catalyst together with an N-substituted cyclic imide compound catalyst (e.g. JP2003-034679 A), is also known. In these conventional methods, however, there are problems that organic compounds of which deterioration caused to decomposition is feared, are allowed to exist, or a solid catalyst difficult in handling is added, therefore, it is difficult to say that they are methods excellent in stability and practicality for obtaining a high oxidation reaction rate.

### SUMMARY OF THE INVENTION

An object of the present invention is to provide a process for producing an alkyl aromatic hydroperoxide by oxidizing an alkyl aromatic compound with an oxygen-containing gas, which can stably obtain the alkyl aromatic hydroperoxide at a high oxidation reaction rate, therefore, is excellent in practicality.

Namely, the present invention relates to a process for producing an alkyl aromatic hydroperoxide, which comprises oxidizing an alkyl aromatic compound with an oxygen-containing gas in the presence of a water-soluble iron compound, wherein a concentration of the iron compound in the oxidation reaction system is 0.0001 to 10 ppm by weight in terms of iron metal.

### DETAILED DESCRIPTION OF THE INVENTION

As the alkyl aromatic compound to be subjected to oxidation, monoalkyl benzenes and dialkyl benzenes are listed. Specific examples of the alkyl aromatic compound include monoalkyl benzenes such as ethylbenzene, cumene and sec-butylbenzene, and dialkyl benzenes such as cymene, m-diisopropyl benzene and p-diisopropyl benzene. Among these, cumene, m-diisopropyl benzene and p-diisopropyl benzene are suitably used.

As the method of oxidizing the alkyl aromatic hydrocarbon with an oxygen-containing gas to obtain an alkyl aromatic hydroperoxide, the following methods are illustrated.

As the oxygen-containing gas, air itself or an oxygen-rich air in which an oxygen concentration was enriched by means of a membrane separation, may be used. Further, a dilute oxygen gas diluted with an inert gas such as nitrogen, argon or helium, may be also used.

In the oxidation reaction, organic acids such as formic acid, acetic acid and the like are produced as by-products with the reaction. When the organic acids as the by-products are produced, there are adverse effects such as a promotion of decomposition of the hydroperoxide as a target product and inhibition of the oxidation reaction caused to products generated by the decomposition, therefore, it is effective to carry out the oxidation reaction in the presence of the aqueous solution to suppress the adverse effects.

An amount of the aqueous solution is usually 0.1 to 20% by weight, preferably 1 to 10% by weight based on an oxidation oil. Herein, the oxidation oil means an oil part of the oxidation reaction mixture composed of the aqueous solution part and the oil part.

When the amount of the aqueous solution is less than 0.1% by weight, the effect of water becomes small, on the other hand, when more than 20% by weight, the productivity reduces because the ratio of the oil layer (the oxidation oil) in the reactor decreases. A pH of the aqueous solution is usually 6 or higher, and preferably 7 to 12. In acidic side of pH of lower than 6, acidolysis of the hydroperoxide may be promoted, and further, corrosion of equipment such as the reactor is feared. On the other hand, when the alkalinity is a pH higher than 12, an alkaline degradation of the hydroperoxide may be unfavorably promoted. For keeping the pH of the aqueous solution within the above range, it is preferable to add an aqueous solution of an alkali metal compound thereto.

As the alkali metal compound, sodium hydroxide, sodium carbonate, sodium hydrogen carbonate or the like is preferably used.

The organic acids such as formic acid, acetic acid and the like produced as by-products in the oxidation reaction, are neutralized with the alkali, and they exist as salts of the organic acids such as sodium formate and sodium acetate.

Existence of such the salts of the organic acids is effective to stably maintain the pH of the aqueous solution within the preferable range. Further, it is preferably conducted to recycle to the oxidation reaction system an aqueous layer containing the organic acids or salts thereof discharged from the reaction system for decreasing an amount of the discharged water. As the aqueous layer containing the organic acids or salts thereof discharged from the reaction system, for example, an aqueous layer obtained by subjecting a reaction mixture to oil-water separation, or an aqueous layer recovered from the oxidation gas purging system. As the result, the concentration of the organic acids salts in the oxidation reaction aqueous solution is usually 0.01 to 50% by weight, preferably 0.1 to 30% by weight.

The greatest characteristic of the present invention is to carry out the oxidation reaction in the presence of a water-soluble iron compound and to keep the concentration of the iron compound within a range of 0.0001 to 10 ppm by weight in terms of iron metal.

Specific examples of the water-soluble iron compound include iron formate, iron acetate, iron propionate, iron hydroxide, iron sulfate, iron nitrate, iron phosphate, iron chloride and iron bromide. Usually, since lower organic acids such as formic acid and acetic acid formed as the products of the oxidation reaction, exist in the oxidation reaction system, it is preferable to allow the iron compound to substantially exist as salts thereof. Further, since iron salts of the lower organic acids are partly dissolved in the oxidation reaction mixture even under a condition of which an aqueous solution is absent, a high oxidation reaction rate can be obtained. Therefore, iron formate and iron acetate as iron salts of the lower organic acids are preferably used. In the present invention, it is not necessary to use a special metal complex. Organic compounds such as amine-type ligands and imide compounds are generally unstable in the oxidation reaction system, and addition of these compounds to the oxidation reaction system leads to an increase of loads of exhaust-gas treatment and wastewater treatment caused to degradative by-production of nitrogen oxides and decomposition products, therefore, it is not preferable.

The concentration of the iron compound in the oxidation reaction system is 0.0001 to 10 ppm by weight, preferably 0.001 to 1 ppm by weight in terms of iron metal. When the concentration is lower than 0.0001 ppm by weight, the effect for heightening the oxidation reaction rate is insufficient, on the other hand, when higher than 10 ppm by weight, it is undesirable in view of loss of the raw material and elevation of a load for purification of the target product because a side-reaction other than the desired oxidation reaction increases.

As an adding method of the iron compound to the oxidation reaction system, it is preferable to supply the water-soluble iron compound as a homogenous aqueous solution obtained by dissolving with water from the viewpoint of stable supply of the iron compound in infinitesimal. As water for dissolving the iron compound, processed water such as water separated from a reaction system, water condensed from an exhausted gas or water recycled from a process, is preferred in the practical viewpoint. Further, as a preferable method of stably dissolving the iron compound of very low concentration with an aqueous solution, there is exemplified a method of stably dissolving an iron-containing metal in trace amount by contacting the metal with water containing an organic acid such as formic acid or acetic acid and/or the hydroperoxide-containing water. In this case, an aqueous solution obtained by dissolving the iron-containing metal outside the reaction system, may be supplied to the oxidation reaction system, further, the iron compound may be generated in the oxidation reaction system by dissolving the iron-containing metal with the aqueous solution inside the oxidation reaction system. Further, as another method of adding the water-soluble iron to the oxidation reaction system, there is exemplified a method of supplying by dissolving the iron compound or the iron-containing metal with the oxidation reaction mixture itself. Usually, in addition to the alkyl aromatic hydroperoxide, a small amount of formic acid or acetic acid also exists in the oxidation reaction mixture, therefore, it is a preferable method to dissolve trace amount of the iron compound by contacting the iron-containing metal with the oxidation reaction mixture.

The iron compound may be added all at once at the initiation of the reaction, continuously added during the reaction, or divisionally added at regular time intervals.

In the present invention, the oxidation reaction is usually carried out at a temperature of 50 to 150° C. under a pressure of 0.1 to 1 MPa. The reaction can be carried out by any one of batch wise and continuous methods.

### EXAMPLES

The present invention will be explained by Examples below.

### Example 1

In to a 1 liter-pressure resistant glass container, 500 g of a cumene solution containing cumene hydroperoxide (CMHP) of 5.2 wt. % and a solution prepared by dissolving iron acetate with 16.7 g of an aqueous solution containing sodium carbonate and sodium salts of an organic acid (containing sodium formate and sodium acetate) were charged. The concentration of iron in the liquid charged was 0.5 wt. ppm in terms of iron metal. The reaction was carried out under flowing of air at 95° C. and 0.7 MPa for 4 hours. The amount of air supplied was controlled so that the oxygen concentration at the outlet of the reactor was kept to 2 vol. % during the reaction. The concentration of cumene hydroperoxide in a reaction mixture after the reaction was 11.6 wt. %.

### Comparative Example 1

A reaction was carried out in the same manner as in Example 1 except that iron acetate was not added. The concentration of cumene hydroperoxide in a reaction mixture after the reaction was 10.9 wt. %.

### Example 2

Into a reactor similar to that used in Example 1, 520 g of an oil containing 25 wt. % of m-diisopropylbenzene, 40 wt. % of m-diisopropylbenzene mono-hydroperoxide as raw materials for reaction and a solution prepared by dissolving iron acetate with 17 g of an aqueous solution containing sodium carbonate and sodium salts of an organic acid (containing sodium formate and sodium acetate) were charged. The concentration of iron in the liquid charged was 0.1 wt. ppm in terms of iron metal. The reaction was carried out under flowing of air at 90° C. and 0.3 MPa for 6 hours. The amount of air supplied was controlled so that the oxygen concentration at the outlet of the reactor was kept to 5 vol. % during the reaction. The increased amount of the concentration of m-diisopropylbenzene dihydroperoxide in the reaction mixture after the reaction, was 6.2 wt. %.

### Comparative Example 1

A reaction was carried out in the same manner as in Example 2 except that iron acetate was not added. The increased amount of the concentration of m-diisopropylbenzene dihydroperoxide in the reaction mixture after the reaction, was 5.9 wt. %.

According to the present invention, a process for producing an alkyl aromatic hydroperoxide by oxidizing an alkyl aromatic compound with an oxygen-containing gas, which can stably obtain an alkyl aromatic hydroperoxide at a high oxidation reaction rate and which is excellent in practicality, can be provided.

1. A process for producing an alkyl aromatic hydroperoxide, which comprises oxidizing an alkyl aromatic compound with an oxygen-containing gas in the presence of a water-soluble iron compound, wherein a concentration of the iron compound in the oxidation reaction system is 0.0001 to 10 ppm by weight in terms of iron metal.

2. The process according to claim 1, wherein the oxidation is carried out in the presence of an aqueous solution of the water-soluble iron compound.

3. The process according to claim 1, wherein the concentration of the iron compound is 0.001 to 1 ppm by weight.

4. The process according to claim 1, wherein the alkyl aromatic compound is a monoalkyl benzene and dialkyl benzene.

5. The process according to claim 1, wherein the alkyl aromatic compound is cumene or diisopropyl benzene.
